(19) United States
(12) Patent Application Publication
(10) Pub. No.: US 2009/0247921 A1
(43) Pub. Date: Oct. 1, 2009

(54) Foot/ankle wrap and method of treating infirmities and abnormalities of the foot and ankle

(76) Inventor: Juli A. Weitzen, Boyertown, PA (US)

Correspondence Address: Zachary T. Wobensmith, III, 7746 101st Court, Vero Beach, FL, 32967-2871, US

(21) Appl. No.: 12/079,554

(22) Filed: Mar. 28, 2008

(51) Int. Cl. A61F 5/00; A61B 19/00

(52) U.S. Cl. 602/27; 128/898

(57) ABSTRACT

A foot/ankle wrap, and method of treating infirmities and abnormalities of the foot or ankle by wrapping the foot or ankle with one or more straps of differing lengths in a predetermined manner, to apply force to treat the infirmity or abnormality. The choice of how to apply the shorter or second strap can be made independently of the choice of the application of the longer or first strap.

### BACKGROUND OF THE INVENTION

1. Field of the Invention

This invention relates to a foot/ankle wrap, and method of treating infirmities and abnormalities of the foot or ankle, by wrapping the foot or ankle with one or more straps in a predetermined manner to apply force thereto to treat the infirmity or abnormality.

2. Description of the Prior Art

It is common practice in the prior art to wrap a foot or ankle with a strap to correct an infirmity or abnormality, but such wraps are limited in that they only correct one type of biomechanical issue.

For example, the prior art wraps can lift the arch and maybe the ankle, but don't have the capability of both lifting the foot and applying the same or an opposite force to the ankle. This is important when supporting a falling arch, but not wanting to contribute to ankle sprain in a patient who has weak ankles, since these prior art wraps or splints raise or lower all of the ankle or foot.

The foot/ankle wrap and method of the invention can be used to control supination, pronation, varus, and valgus. Pronation and supination will be used in describing corrections by using a first strap. Valgus, Varus, and Neutral will be used in describing corrections obtained by using a second strap.

### SUMMARY OF THE INVENTION

This invention relates to a foot/ankle wrap, and method of applying a wrap to a foot or ankle to control, supination, pronation, varus, valgus, apply a neutral force to the foot or ankle, and for arch support.

The principal object of the invention is to provide a versatile foot/ankle wrap, and a method of treating infirmities or abnormalities of the foot and/or ankle by wrapping the foot or ankle in a predetermined manner to treat or correct the infirmity or abnormality.

A further object of the invention is to provide a foot/ankle wrap, and method which can be applied by a patient, or a health care practitioner

A further object of the invention is to provide a foot/ankle wrap, and method, which is easy to understand and use.

A further object of the invention is to provide a foot/ankle wrap, and method, which can be used to treat or correct any biomechanical issue of the foot or ankle.

Other objects and advantageous features of the invention will be apparent from the description and claims.

### DESCRIPTION OF THE DRAWINGS

The nature and characteristic features of the invention will be more readily understood from the following description taken in connection with the accompanying drawings forming part hereof in which:

FIG. 1A is a plan view illustrating the foot wrap, and method of the invention for applying a force of supination to control and resist pronation in a left foot;

FIG. 1B is a view similar to FIG. 1A but for the right foot.

FIG. 2A is a plan view illustrating the foot/ankle wrap, and method of applying a force of pronation to control and resist supination in a left foot.

FIG. 2B is a view similar to FIG. 2A, but for the right foot.

FIG. 3 is a plan view illustrating the addition of a second strap to the foot/ankle wrap, with the method of applying a force to control and lessen valgus in a left foot.

FIG. 4 is a plan view illustrating the addition of a second strap to the foot/ankle wrap, with the method of applying a force to control and lessen varus in a left foot.

FIG. 5 is a plan view illustrating the addition of a second strap to the foot/ankle wrap, with the method of applying a neutral force to the ankle of a left foot.

FIG. 6 is a plan view illustrating the addition of a second strap to the foot/ankle wrap, with the method of foot/ankle control and stabilization with the center of the second strap of the wrap centered at the Achilles location, and

FIG. 7 is a plan view illustrating an optional thermal moldable orthotic insert, that can be used with the foot/ankle wrap of the invention.

It should, of course, be understood that the description and drawings herein are merely illustrative and that various modifications and changes can be in the structures disclosed without departing from the spirit of the invention.

Like numerals refer to like parts throughout the several views.

### DESCRIPTION OF THE PREFERRED EMBODIMENTS

When referring to the preferred embodiments, certain terminology will be utilized for the sake of clarity. Use of such terminology is intended to encompass not only the described embodiment, but also technical equivalents, which operate and function in substantially the same way to bring about the same result.

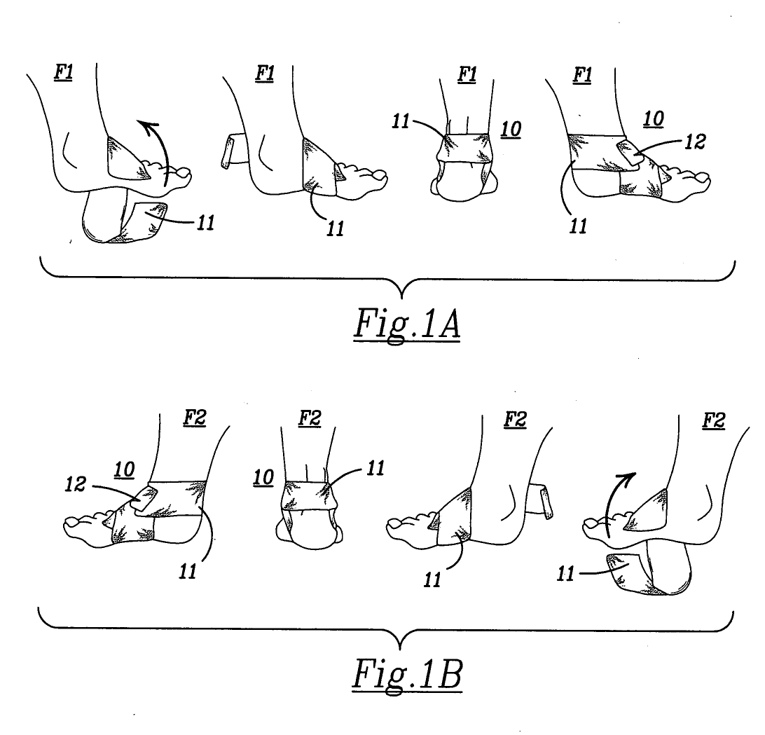

Referring now more particularly to the drawings and FIGS. 1A and 1B of the drawings, a typical left foot F, and a right foot F1 are shown. One embodiment of a foot/ankle wrap 10 is illustrated, which consists of a long length strap 11 and a plurality of Velcro clips 12 of well-known type. The long strap 11 is composed of any well known material, which is compatible with the Velcro clips 12 for engagement and fastening of the strap 11, with “Fabrifoam”, available from Fabrifoam Products, Exton Pa., 19341, being particularly suitable.

The feet F and F1 as illustrated have pronation, i.e. in that they are downwardly rotated, and for correction a force of supination, or upward force has to be applied to the feet F or F1. To apply the wrap 10, the long strap 11 is started on the dorsal mid foot, and then directed laterally and then plantarly, and continued in a circular direction dorsally to overlap the starting position of strap 11. Continue to wrap the feet F and F1 with the strap 11 to encompass the lateral malleolus, and wrap it posteriorly, then encompass the medial malleolus, and continue across the anterior ankle, and fasten it with a Velcro clip 12. During the strap 11 application the patient (not shown) should be encouraged to dorsiflex and supinate the foot F, or F1 being treated.

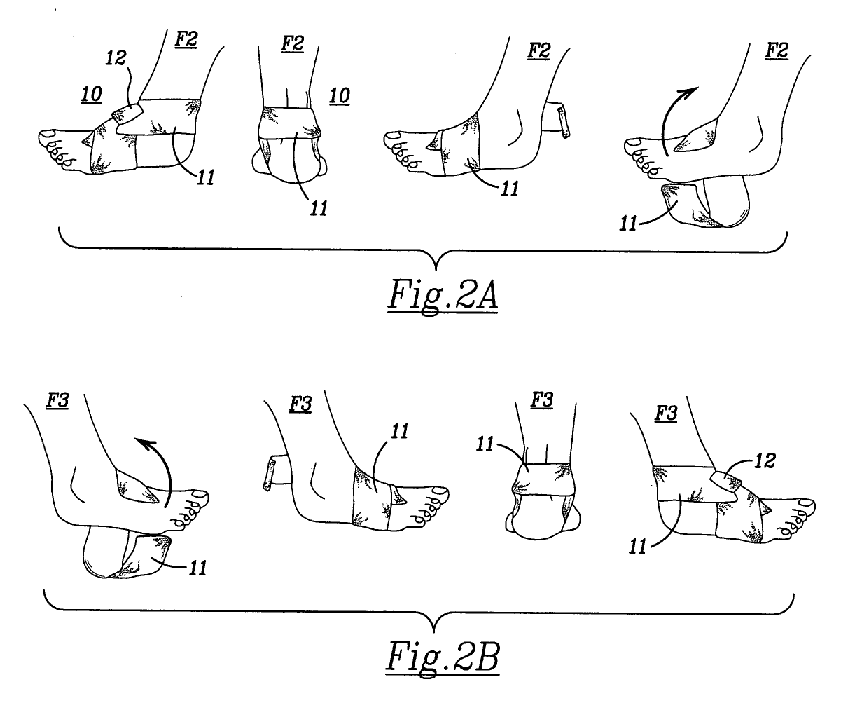

Referring now to FIGS. 2A and 2B, feet F2 and 3 are shown, which as illustrated have supination, i.e. in that they are upwardly rotated, and for correction, a force of pronation or downward (flattening) force has to be applied to foot F2 or F3.

To apply wrap 10, the long strap 11 is started on the dorsal midfoot of foot F2, or F3, directed medially and then plantarly, and continued in a circular direction dorsally to overlap the starting position of strap 11.

Continue to wrap the strap 11 to encompass the medial malleolus, wrap it posteriorly, and then encompass the lateral malleolus, continue across just below the anterior ankle, and fasten with a Velcro clip 12. During the strap application, the patient (not shown) should be encouraged to dorsiflex and pronate the foot F2, or F3 being treated.

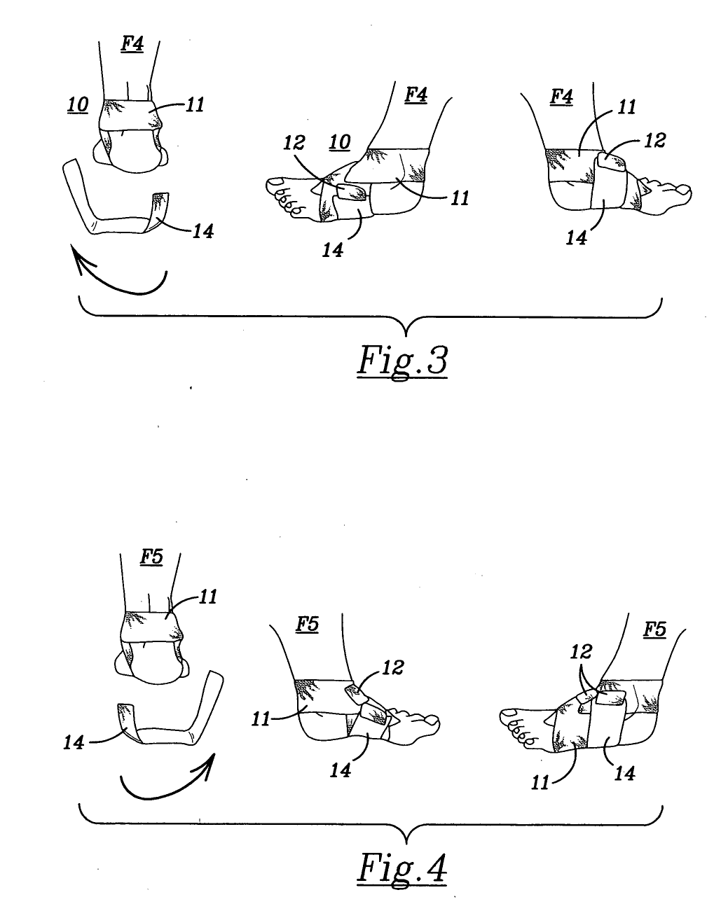

Referring now to FIG. 3, a foot F4 is illustrated, which is bent or turned outwardly i.e. in a valgus condition. For correction a force of varus must be applied for correction, with the long strap 11 applied as described for FIG. 1A, 1B, 2A, or 2B. A short strap 14 is selected and attached to long strap 11 on the lateral side by a Velcro strip 12, with the short strap 14 wrapped laterally and inferior to the arch of the foot F4, directed supromedially continuing across the anterior ankle, and attached to the long strap 11 at the top of its medial aspect by a Velcro clip 12. The strap 14 is preferably of the same material as strap 11.

Referring now to FIG. 4, a foot F5 is illustrated, which is bent or turned inwardly, i.e. in a varus condition. A long strap 11 is applied as described for FIG. 1A, 1B, 2A, or 2B. A short strap 14 is attached to the long strap 11 by Velcro clip 12 on the medial side, and wrapped inferior to the arch, continued suprolaterally, and continued across the anterior ankle, and attached to long strap 11 at the top of the lateral aspect of strap 11 by a Velcro clip 12.

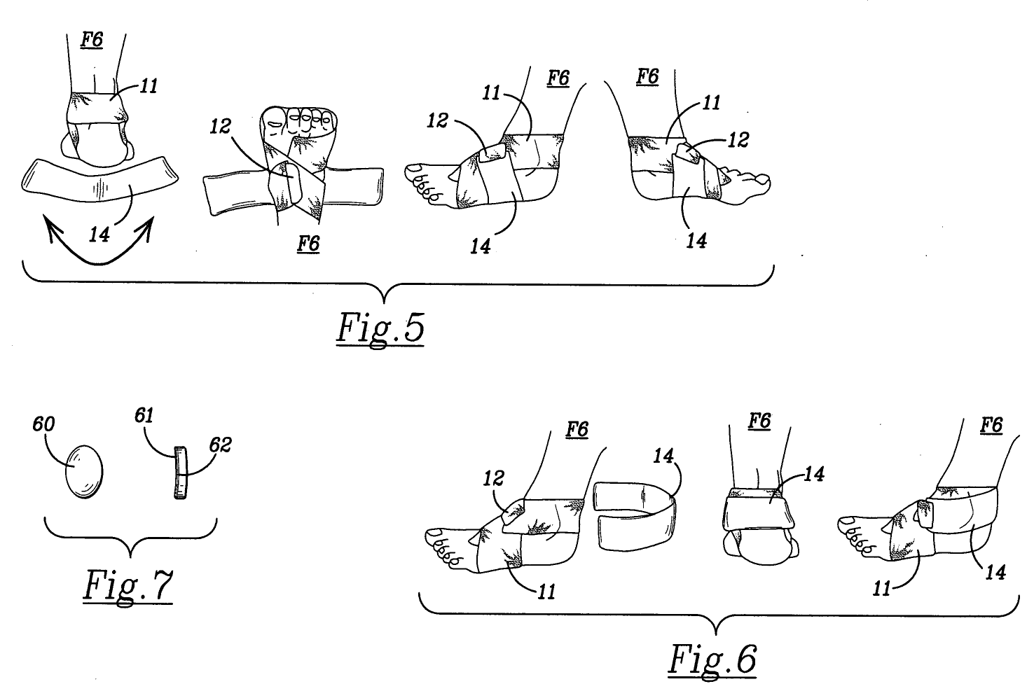

Referring now to FIG. 5, wherein it is desired to apply a neutral force to the ankle of a foot F6. The long strap 11 is applied as shown in FIG. 1A, 1B, 2A, or 2B.

A short strap 14 is applied at the center of the arch, equal tension is placed on each end of strap 14, and it is fastened to long strap 11 by Velcro clips 12 at each end, which are directed towards the front of the ankle.

Referring now to FIG. 6, wherein it is desired to apply a neutral force to the foot/ankle, strap 11 is applied as shown in FIG. 1A, 1B, 2A, or 2B, a short strap 14 is applied around the ankle centered at the Achilles, with equal tension applied to both ends, anteriorly and slightly superiorly along the ankle ligaments toward the anterior ankle, and fastened with a Velcro clip 12.

If arch support (not shown) is desired, a long strap 11 is applied as shown in FIG. 1A, 1B, 2A, or 2B, a short strap 14 is applied centered at the plantar arch, with equal tension applied to both ends to envelop the arch, and fastened at the dorsal foot with a Velcro clip 12.

Referring now to FIG. 7, a thermal moldable plastic orthotic insert 60 is illustrated, which is shown of oval configuration, but could be of other configurations if desired. The insert 60 can have a layer of foam cushion 61, and a layer of adhesive 62 if desired. The insert 60 is inserted between the straps 11, or 14 and the foot or ankle of the patient. The insert 60 can molded by applying heat thereto to cause it to conform to the contour of the patients foot/ankle, to provide additional support. Examples of applications are for one insert at the medial malleolus, and/or one at the lateral malleolus, and/or one at the arch.

It will thus be seen that foot/ankle wraps, and methods of applying them, have been described with which the objects of the invention are attained.

I claim:

1. A foot/ankle wrap for applying a force to the foot or ankle of a person which comprises;
a first strap to be wound around said foot/ankle and connected by clip means in a manner to correct biomechanical issues of the person's foot or ankle.

2. A foot/ankle wrap as defined in claim 1 in which;
a second strap is provided of a shorter length than said first strap, which is also wound around said foot/ankle and connected by clip means to said first strap in a manner to correct biomechanical issues of the person's foot, or ankle.

3. A foot/ankle wrap as defined in claims 1 or 2 in which;
said first strap and said second strap are of a fabric material, and fastened by said clip means.

4. A method of treating infirmities or abnormalities of the foot or ankle of a person wherein it is desired to treat pronation, which comprises;
applying a force of supination to said foot or ankle by wrapping a first strap of fabric around said foot starting on the dorsal mid foot, then directed laterally and plantarly in a circular direction to dorsally overlap the starting position;
continuing to wrap the foot to encompass the lateral malleolus and then directed posteriorly, to encompass the medial malleolus, and then to cross the anterior ankle, and applying at least one clip means to fasten said strap together.

5. A method of treating infirmities or abnormalities of the foot or ankle of a person wherein it is desired to treat supination which comprises;
applying a force of pronation to said foot or ankle by wrapping a first strap of fabric around said foot starting at the dorsal midfoot, continuing medially, and then plantarly in a circular direction dorsally to overlap the starting position;
continuing to wrap said strap to encompass the medial malleolus, and then directed posteriorly and then to encompass the lateral malleolus and then to cross just below the anterior ankle, and
applying at least one clip means to fasten said strap together.

6. A method of treating infirmities or abnormalities of the foot or ankle of a person wherein it is desired to treat foot pronation, and ankle valgus which comprises;
applying a force of supination to said foot or ankle by wrapping said foot with a first strap,
applying a force of ankle varus by attaching a second strap to said first strap on the lateral side of said foot by a clip means;
wrapping said second strap laterally and inferior to the arch of the foot, and directed supromedially to cross the anterior ankle and,
applying a clip means to said first strap and said second strap at the top of the anterior aspect of said first strap.

7. A method of treating infirmities or abnormalities of the foot or ankle of a person wherein it is desired to treat foot supination, and ankle varus which comprises;
applying a force of pronation to said foot or ankle by wrapping said foot with a first strap,
attaching a second strap to said first strap by a connecting clip means on the medial side of said foot;
wrapping said foot inferior to the arch, and continuing suprolaterally across the anterior ankle, and
applying a clip means to said first strap and said second strap at the top of the anterior aspect of said first strap.

8. A method of treating infirmities and abnormalities of the foot or ankle of a person, wherein it is desired to apply a neutral force to the ankle of said person which comprises;
applying a first strap to said foot/ankle, and;
applying the center of a second strap to the center of the plantar arch of said foot;
applying equal tension to the ends of said second strap and;
applying a clips means to each end of said second strap which is directed towards the front of the ankle.

9. A method of treating infirmities or abnormalities of the foot/ankle of a person wherein it is desired to apply a neutral force to said foot/ankle, which comprises;
applying a first strap to said foot/ankle;
applying a second strap to said ankle centered at the Achilles, with equal tension applied to both ends of said second strap anteriorly and slightly superiorly along the ankle ligaments and toward the anterior ankle, and
applying at least one clip means to said second strap to fasten it together.

10. A method of treating infirmities or abnormalities of the foot or ankle of a person wherein it is desired to provide arch support to said foot or ankle, which comprises;
applying a first strap to said foot or ankle;
applying a second strap centered at the plantar arch to envelop the arch, with equal tension applied to both ends of said second strap, and
applying at least one clip means to said second strap to fasten it together.

11. A method of treating abnormalities of the foot or ankle of a person as defined in claim 2 in which; at least one thermal moldable insert means is inserted between one or both straps and the foot or ankle of the person being treated.
